(19) United States
(12) Patent Application Publication
(10) Pub. No.: US 2011/0201828 A1
(43) Pub. Date: Aug. 18, 2011

(54) METHOD AND CATALYSTS FOR PRODUCING CYCLIC CARBONATES

(75) Inventors: Roman Prochazka, Mannheim (DE); Veronika Wloka, Mannheim (DE)

(73) Assignee: BASF SE, Ludwigshafen (DE)

(21) Appl. No.: 13/124,138

(22) PCT Filed: Oct. 12, 2009
(86) PCT No.: PCT/EP2009/063249

§ 371 Date: Apr. 14, 2011

(30) Foreign Application Priority Data
Oct. 17, 2008 (EP) 08166949.1

(51) Int. Cl. C07D 317/34; B01J 21/06

(52) U.S. Cl. 549/229; 549/228; 502/340

(57) ABSTRACT

Process for the preparation of cyclic carbonates by reacting a polyhydric alcohol with a dialkyl carbonate in the presence of a heterogeneous catalyst, wherein the heterogeneous catalyst is a basic mixed oxide or a basic oxide applied to a support.

The invention relates to a process for the preparation of cyclic carbonates by reacting a polyhydric alcohol with a dialkyl carbonate in the presence of a heterogeneous catalyst wherein the heterogeneous catalyst is a basic mixed oxide or a basic oxide applied to a support.

Cyclic carbonates, in particular glycerol carbonate, are of importance as solvents, additives for cosmetics and cleaning compositions. They are also used for the preparation of epoxy resins, polycarbonates and polyurethanes.

Cyclic carbonates such as glycerol carbonate can be prepared by reacting polyhydric alcohols with dialkyl carbonate in the presence of a catalyst.

The use of homogeneous and heterogeneous catalysts is known.

According to U.S. Pat. No. 5,091,543, alkylammonium salts or pyridinium salts are used as homogeneous catalysts.

EP-A 739 888 discloses the use of zeolites as catalysts. The reaction mixture of glycerol and dialkyl carbonate comprises the zeolites as heterogeneous catalysts.

DE-A 10 2005 060 732 describes the use of basic catalysts. The basic catalysts are in particular alkali metal and/or alkaline earth metal salts, e.g. the corresponding oxides, hydroxides or chlorides. Preference is given to using mixtures of chlorides and oxides. The alkali metal and alkaline earth metal chlorides listed, in particular lithium chloride, are at least partially water-soluble.

Homogeneous catalysts fundamentally have the disadvantage that they can only be separated off from the product mixture with difficulty. By contrast, heterogeneous catalysts can be isolated again easily and be reused.

For the above reaction, therefore, heterogeneous catalysts are desired which bring about the highest possible yield and selectivity for the shortest possible reaction time. Furthermore, the heterogeneous catalyst should comprise as few constituents as possible which dissolve (leach) out of the catalyst under the reaction conditions and thus discharge undesired impurities into the product mixture. Particularly during the work-up, e.g. of glycerol carbonate, the presence of such impurities can be critical and during the distillation of glycerol carbonate can cause a decomposition to give explosive glycidol. Moreover, the required amounts of catalyst should be as low as possible.

An object of the present invention was therefore a process for the preparation of cyclic carbonates in which catalysts of this type are used.

Accordingly, the process defined at the start has been found.

In the process according to the invention, cyclic carbonates are prepared by reacting a polyhydric alcohol with a dialkyl carbonate.

Illustrated using the example of glycerol and dimethyl carbonate, the reaction proceeds according to the following equation:

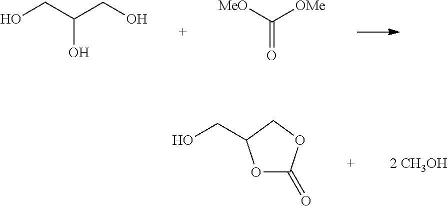

### Starting Materials

Suitable polyhydric alcohols are chemical compounds having at least two hydroxyl groups which form a ring system through reaction with dialkyl carbonates.

Preferred polyhydric alcohols are aliphatic compounds having at least two hydroxyl groups which are in the 1,2 position, 1,3 position and 1,4 position relative to one another. In the 1,2 position, the two hydroxyl groups are located on adjacent carbon atoms and in the above reaction a 5-membered ring is formed; in the 1,3 position a 6-membered ring is correspondingly formed and in the case of a 1,4 position, a 7-membered ring. Preference is given to a 1,2 position, with the formation of a 5-membered ring.

The polyhydric alcohol is preferably an aliphatic diol or triol in which two hydroxyl groups are in the 1,2 position. The polyhydric alcohol preferably comprises no further functional groups and in particular has a molecular weight of less than 300 g/mol.

It is particularly preferably glycerol.

Suitable dialkyl carbonates are, for example, carbonates with C1 to C10 alkyl groups; in particular, dimethyl carbonate or diethyl carbonate may be mentioned.

Particular preference is given to dimethyl carbonate.

### Heterogeneous Catalyst

A heterogeneous catalyst is used according to the invention. The heterogeneous catalyst forms a separate phase under the reaction conditions. In the present case, the heterogeneous catalyst is solid, whereas the starting materials are liquid or gaseous.

The heterogeneous catalyst used is a basic mixed oxide or a basic oxide applied to a support.

The mixed oxide is an oxide of at least two different elements. The mixed oxide is basic if it leads to a pH increase in water.

The mixed oxides are preferably mixtures of at least two oxides selected from oxides of metals of main groups I a, II a, III a, and IV a of the Periodic Table of the Elements, of sub-groups II b, III b and IV b of the Periodic Table of the Elements, and also of the lanthanides. The oxides are selected according to the extent that the resulting mixed oxide is basic.

Preferably, for this, the mixed oxide comprises in particular at least one oxide from main group II a; however, basic mixed oxides without co-use of an oxide from group II a are also possible, a mixed oxide of zinc oxide (ZnO) and aluminum oxide (Al₂O₃), for example, being suitable.

Particular preference is given to mixed oxides which are composed of oxides of metals of groups I a, II a, Ill a, I b and II b of the Periodic Table of the Elements, where at least one oxide of a metal of main group II a is present in the mixed oxide.

The mixed oxide can comprise more than two metal oxides, although a large number of different metal oxides in the mixed oxide is not necessary. Even mixed oxides of two or three metal oxides, in particular of two metal oxides, are highly suitable.

In one preferred embodiment, the heterogeneous catalyst is a basic oxide applied to a support.

The support can be made of any desired inorganic material, mention being made, for example, of zeolites, carbons, polymers, aluminum oxide, titanium oxide, zirconium oxide, silicon dioxide, magnesium oxide, silicon dioxide-aluminum oxides, silicon dioxide-titanium dioxide, silicon dioxide-zirconium dioxide, titanium dioxide-zirconium dioxide, magnesium oxide-aluminum oxides.

The support is preferably an inorganic support, in particular a metal oxide or a non-metal oxide.

In particular, it is a support made of zirconium oxide, silicon oxide, aluminum oxide, titanium oxide or mixtures thereof.

Zirconium oxide is very particularly preferred.

At least one basic oxide is applied to the support. This may be, for example, a metal oxide of metals of main groups I a, II a, III a, and IV a of the Periodic Table of the Elements, of sub-groups II b, III b and IV b of the Periodic Table of the Elements, and also of the lanthanides.

It is also possible to apply two or more oxides, in particular also the mixed oxides listed above, to the support.

Preferably, at least one metal oxide of a metal of the main group I a or II a, of sub-group III b or of the lanthanides is applied to the support.

It is particularly preferably an alkaline earth metal oxide (main group II a), e.g. CaO, BaO or MgO.

Very particular preference is given to CaO.

In a very particularly preferred embodiment, the heterogeneous catalyst is a supported catalyst made of a metal oxide, in particular ZrO₂, TiO₂, SiO₂ or Al₂O₃, particularly preferably ZrO₂, to which at least one further metal oxide, in particular an alkaline earth metal oxide, e.g. CaO, BaO or MgO, is applied. In particular, it is a basic supported catalyst with a support made of zirconium dioxide to which CaO is applied.

The applied metal oxide can be located for the large part on the surface of the support, although the metal oxide, or the corresponding metal cations, can also be distributed largely uniformly within the support; the person skilled in the art also talks here of a doping of the oxidic support with the metal cations in question. The method of application or distribution in or on the support is of secondary importance for the effect of the catalyst and depends only on the preparation process and/or the porosity of the support.

The content of applied metal oxide in the supported catalyst corresponds preferably to an amount of from 0.1 to 25% by weight, particularly preferably from 0.5 to 10% by weight, of alkaline earth metal cations, based on the total supported catalyst.

The catalysts may additionally be modified, e.g. through a further doping with other alkali metals, alkaline earth metals, chalcogens or halogens. However, they preferably comprise no or at most low constituents of compounds which are able to dissolve out of the heterogeneous catalyst under the reaction conditions and thus lead to the problem of “leaching”.

In particular, therefore, a content of water-soluble alkali metal salts, such as LiCl, is as low as possible. Preferably, the content of water-soluble alkali metal salts is less than 0.5 part by weight, particularly preferably less than 0.1 part by weight, per 100 parts by weight of heterogeneous catalyst.

The heterogeneous catalysts may be used in the form of powders or preferably in the form of moldings such as extrudates, chips, rings, hollow cylinders, beads or tablets with a characteristic diameter of from 0.1 to 5 mm, preferably 1 to 3 mm. The characteristic diameter arises here from the sextuple of the quotient of the molding volume and the geometric molding surface. To prepare moldings, binders may be added to the catalyst.

The catalysts can have pores, e.g. with a pore volume 0.05 to 1.0 ml/g.

### Preparation of the Heterogeneous Catalysts

The basic mixed oxides can be prepared by customary methods known to the person skilled in the art.

To prepare the basic mixed oxides, the desired metal salts can firstly be dissolved in water and then be precipitated out using a suitable precipitant (e.g. aqueous ammonia solution, solutions of alkali metal carbonates or hydrogen carbonates, such as, for example, sodium carbonate, urotropin, etc.). The resulting solid can then be washed and dried, e.g. including by spray-drying. Finally, the product obtained can be activated immediately or only briefly prior to subsequent use at temperatures of from 200 to 1200, in particular 400 to 600° C., e.g. under air or nitrogen.

The preparation of the basic supported catalyst can also take place in accordance with customary processes known to the person skilled in the art.

Here too, preferably an aqueous solution of the desired metal salt is firstly prepared. The support is then treated with this solution. The amount of solution here can be measured such that it is absorbed completely or virtually completely by the support (saturation to water absorption). However, the catalyst can also be dispersed in the aqueous solution and then be separated off from the solution, e.g. by filtration. Subsequently, activation at high temperatures, as described above, can again take place.

### The Process

The starting materials can be reacted in the liquid or gas phase in the presence of the heterogeneous catalyst.

In the case of the above starting materials, in particular glycerol and dimethyl carbonate, the reaction preferably takes place in the liquid phase.

The polyhydrate alcohol or the dialkyl carbonate can be used in excess. In one preferred embodiment, the dialkyl carbonate, preferably dimethyl carbonate, is used in excess, e.g. 1.1 to 10 mol, in particular 1.5 to 8 mol or 1.5 to 5 mol, of dialkyl carbonate are used per 1 mol of polyhydrate alcohol (glycerol).

The reaction can take place at atmospheric pressure, reduced pressure or super-atmospheric pressure. It is preferably carried out at atmospheric pressure.

The reaction is preferably carried out at elevated temperature, e.g. at temperatures of from 30 to 100° C., in particular 50 to 90° C.

The reaction can be carried out discontinuously (batchwise, i.e. with initial charge of the total amount of all starting materials), semicontinuously (metered addition of some of the starting materials during the reaction) or continuously. In the case of the continuous procedure, both the polyhydrate alcohol and also the dialkyl carbonate are preferably introduced continuously.

The heterogeneous catalyst is preferably used in amounts of from 0.1 to 10 parts by weight, particularly preferably 0.2 to 5 parts by weight, based on 100 parts by weight of polyhydric alcohol.

The process according to the invention achieves a high yield and selectivity of cyclic carbonate for short reaction times. The required amount of the heterogeneous catalyst is low.

The heterogeneous catalyst can be separated off from the product mixture in a simple manner, e.g. by filtration, optionally worked up and reused. A content of soluble constituents in the heterogeneous catalyst which leads to the problem of “leaching” can be dispensed with.

The heterogeneous catalyst also has a high useful life. In the case of the continuous procedure, a high yield and selectivity is maintained over a long period.

In the case of a discontinuous procedure (batch preparation), the catalyst can be used repeatedly; after the batch preparation of the cyclic carbonate is complete, the catalyst can be separated off from the product and be used again for a new batch preparation; this can be repeated several times, e.g. up to 10 times, with reuse of the same catalyst.

### EXAMPLES

### A) Preparation of the Catalysts

An overview of the prepared catalysts is given in table 1.

### Example 1

10 l of water were initially introduced in a precipitation vessel and then simultaneously a 20% strength aqueous Na₂CO₃ solution and an aqueous solution of calcium nitrate and aluminum nitrate (134 kg of solution, comprising 4.81 kg of CaO and 8.19 kg of Al₂O₃) were precipitated with stirring at 80° C. and pH 5.5. After adding a total of 190 kg of Na₂CO₃ solution, the end pH was 7.8. The product was filtered, washed with water and dried for 16 h at 100° C. 19.06 kg of the dried product were kneaded with 10.69 l of water and 190 g of polyethylene oxide and extruded to give 1.5 mm extrudates. The extrudates were dried for 16 h at 120° C. and finally calcined in air at 600° C. for 1 h. 15.1 kg of extrudates were obtained with a CaO content of 35% and an Al₂O₃ content of 65%.

### Example 2

10 l of water were initially introduced in a precipitation vessel and then simultaneously a 20% strength aqueous Na₂CO₃ solution and an aqueous solution of magnesium nitrate, zinc nitrate and aluminum nitrate (140 kg of solution, comprising 1.56 kg of MgO, 3.25 kg of ZnO and 8.19 kg of Al₂O₃) were precipitated with stirring at 80° C. and pH 5.5. After adding a total of 190 kg of Na₂CO₃ solution, the end pH was 7.8. The product was filtered, washed with water and dried for 16 h at 100° C. 18.62 kg of the dried product were kneaded with 4.8 l of water and extruded to give 1.5 mm extrudates. The extrudates were then dried for 16 h at 120° C. and finally calcined in air at 600° C. for 1 h. 12.1 kg of extrudates were obtained with an MgO content of 11%, a ZnO content of 25% and an Al₂O₃ content of 64%.

### Example 3

150 kg of zirconium oxide (Material D9-89, BASF) were kneaded with 16.65 kg of 70% strength Silres MSE 100 (Wacker), 35 l of water and 1.5 kg of polyethylene oxide for 60 min. The material was then extruded to give 1.5 mm extrudates, which were dried overnight on a belt calciner at 120° C. and then calcined at 580° C. for 2 h. 143.8 kg of white zirconium oxide extrudates were obtained.

### Example 4

120 g of zirconium oxide extrudates from example 3 were placed into a flask and a clear solution of 29.5 g of Ca(NO₃)₂*4 H₂O and 38 g of water was added. The material was a number of times thoroughly mixed well, then dried in air at 120° C. for 16 h and finally calcined in air at a heating rate of 10 K/min at 500° C. for 2 h. 123.6 g of white extrudates with a Ca content of 3.5% were obtained.

### Example 5

200 g of zirconium oxide (Material SZ 31108, Norpro) in the form of 3 mm extrudates were calcined in air for 5 h at 500° C. The extrudates were placed into a flask and a clear solution of 93.74 g of Ca(NO₃)₂*4 H₂O and 80.4 g of water were added. The impregnated extrudates were then predried for 30 min at RT and for a further 30 min at 80° C. on a rotary evaporator. The extrudates were then dried in air at 100° C. for 16 h and finally calcined in air at a heating rate of 2 K/min at 500° C. for 16 h. 226.9 g of beige-colored extrudates with a Ca content of 6.5% were obtained.

### Example 6

200 g of zirconium oxide (Material SZ 31108, Norpro) in the form of 3 mm extrudates were calcined in air for 5 h at 500° C. The extrudates were placed into a flask and a clear solution of 155.44 g of Mg(NO₃)₂*6 H₂O and 80.4 g of water was added. The impregnated extrudates were then predried for 30 min at RT and for a further 30 min at 80° C. on a rotary evaporator. The extrudates were then dried in air at 100° C. for 16 h and finally calcined in air at a heating rate of 2 K/min at 500° C. for 16 h. 206.4 g of white extrudates with a Mg content of 1.7% were obtained.

### Example 7

120 g of titanium oxide (Material S 150, Finnti) in the form of 1.5 mm extrudates were placed into a flask and a clear solution of 29.5 g of Ca(NO₃)₂*4 H₂O and 62 g of water was added. The material was thoroughly mixed well several times, then dried in air at 120° C. for 16 h and finally calcined in air at a heating rate of 10 K/min at 500° C. for 2 h. 124.2 g of white extrudates with a Ca content of 4.2% were obtained.

### Example 8

120 g of silicon dioxide (Material D11-10, BASF) in the form of 1.5 mm extrudates were placed into a flask and a clear solution of 29.5 g of Ca(NO₃)₂*4 H₂O and 166 g of water was added. The material was thoroughly mixed well several times, then dried in air at 120° C. for 16 h and finally calcined in air at a heating rate of 10 K/min at 500° C. for 2 h. 124.5 g of white extrudates with a Ca content of 4.2% were obtained.

### Example 9

120 g of aluminum oxide (Material D10-10, BASF) in the form of 1.5 mm extrudates were placed into a flask and a clear solution of 29.5 g of Ca(NO₃)₂*4 H₂O and 144 g of water was added. The material was thoroughly mixed well several times, then dried in air at 120° C. for 16 h and finally calcined in air at a heating rate of 10 K/min at 500° C. for 2 h. 123.8 g of white extrudates with a Ca content of 3.3% were obtained.

### Example 10

2.6 kg of calcium carbonate were calcined at 800° C. for 2 h and further processed under nitrogen. 1.4 kg of the resulting CaO powder were mixed with 42 g of magnesium stearate. The mixture was processed on a rotary tabletting machine to give 5×3 mm tablets with a pressing force of 25 kN. The tablets were then calcined at 450° C. in air for 2 h.

### Example 11

1.5 kg of MgO granules (Magnesia) were mixed with 45 g of magnesium stearate. The mixture was processed on a rotary tabletting machine to give 3×3 mm tablets with a pressing force of 12.4 kN. The tablets were finally calcined at 450° C. in air for 2 h.

### B) Example for the Preparation of Glycerol Carbonate

The experimental procedure was carried out in stirred glass apparatuses with water separator under reflux.

### Standard Batch:

23 g (0.25 mol) of glycerol (anhydrous, high-purity; >99%, Merck), 90 g (1 mol) of dimethyl carbonate (99%, ACROS) and 0.6 g of catalyst (=2.6% by weight with regard to glycerol) were initially introduced into a catalyst cage in a 4-neck flask equipped with a stirrer and a reflux condenser. The reaction mixture was heated to 80° C. The reaction progress was monitored by means of GC (30 m DB5 column). After the reaction was complete, the reaction mixture was cooled and the catalyst was separated off. The low-boiling components were then distilled off in vacuo and the crude product was obtained.

TABLE 1
Experimental series with Mg, Ca and Ba catalysts

| | | Time | C | S | Y |
|---|---|---|---|---|---|
| Cat. No. | Catalyst | [h] | [%] | [%] | [%] |
| 1 | CaO—Al₂O₃ | 15 | 100 | 73.0 | 73.0 |
| 2 | MgO—ZnO—Al₂O₃ | 14 | 64.0 | 80.0 | 51.2 |
| 4 | CaO/ZrO₂ | 4 | 90.2 | 98.7 | 89.0 |
| 5 | CaO/ZrO₂ | 21 | 98.5 | 93.6 | 92.2 |
| 6 | MgO/ZrO₂ | 21 | 78.5 | 99.0 | 77.7 |
| 7 | CaO/TiO₂ | 13 | 100 | 63.4 | 63.4 |
| 8 | CaO/SiO₂ | 15 | 79.0 | 97.6 | 77.1 |
| 9 | CaO/Al₂O₃ | 13 | 100 | 67.9 | 67.9 |
| 10 | CaO | 19 | 100 | 62 | 62 |
| 11 | MgO | 13 | 71 | 100 | 71 |

Catalysts 1 and 2 are mixed oxides according to the invention. Catalysts 4 to 9 are supported catalysts according to the invention, catalysts 10 and 11 are for comparison.

### Catalyst Leaching

In order to be able to compare the catalyst stability, experiments were carried out using the catalysts described in the application by Rohm GmbH (DE 10 2005 060 732) and the above-described catalyst No. 4. The catalysts from DE 10 2005 060 732 are a dried and/or calcined mixture of CaO and LiCl; in both cases, severe leaching was observed. By contrast, catalyst 4 comprises neither alkali metal doping nor halides and has no significant leaching (Tab. 2).

TABLE 2
Comparison of catalyst leaching

| Catalyst | Ca [ppm] | Li [ppm] | Cl [ppm] | Ti (ppm) |
|---|---|---|---|---|
| CaO/LiCl dried | 2800 | 610 | 2600 | — |
| CaO/LiCl calcined at | 4 | 380 | 1900 | — |
| 500° C. | | | | |
| Catalyst 4 | 14 | — | — | <1 |

1. A process for preparing at least one cyclic carbonate, the process comprising:
reacting a polyhydric alcohol with a dialkyl carbonate in the presence of a heterogeneous catalyst,
wherein the heterogeneous catalyst is a basic mixed oxide or a basic oxide applied to a support.

2. The process of claim 1, wherein glycerol is reacted with a dialkyl carbonate to give glycerol carbonate.

3. The process of claim 1, wherein the dialkyl carbonate is dimethyl carbonate.

4. The process of claim 1, wherein the heterogeneous catalyst is a basic oxide applied to a support.

5. The process of claim 4, wherein the support comprises at least one selected from the group consisting of zirconium oxide, silicon oxide, aluminum oxide, and titanium oxide.

6. The process of claim 4, wherein the support is a support comprising zirconium oxide.

7. The process of claim 4, wherein the basic oxide is an alkaline earth metal oxide.

8. The process of claim 4, wherein the basic oxide is calcium oxide.

9. The process of claim 4, wherein the heterogeneous catalyst comprises less than 0.1 part by weight of water-soluble alkali metal salt per 100 parts by weight of heterogeneous catalyst.

10. The process of claim 1, wherein the heterogeneous catalyst is present in amounts of from 0.1 to 10 parts by weight, based on 100 parts by weight of polyhydric alcohol.

11. A cyclic carbonate, obtained by the process of claim 1.

12. A solvent, an additive in cosmetics, a cleaning composition, or a starting material for preparing an epoxy resin, a polycarbonate, or a polyurethane, comprising the cyclic carbonate of claim 11.

13. A supported catalyst, comprising a support comprising zirconium dioxide and a content of at least one alkaline earth metal oxide.

14. A supported catalyst, comprising with a support comprising zirconium dioxide and a content of calcium oxide.

15. The supported catalyst of claim 13, wherein the content of alkaline earth metal cations is 0.1 to 25% by weight, based on a total weight of the supported catalyst.

16. The process of claim 2, wherein the dialkyl carbonate is dimethyl carbonate.

17. The process of claim 2, wherein the heterogeneous catalyst is a basic oxide applied to a support.

18. The process of claim 2, wherein the heterogeneous catalyst is a basic oxide applied to a support.

19. The process of claim 4, wherein the support consists essentially of at least one selected from the group consisting of zirconium oxide, silicon oxide, aluminum oxide, and titanium oxide.

20. The process of claim 4, wherein the support consists essentially of zirconium oxide.
